(19) United States
(12) Patent Application Publication
(10) Pub. No.: US 2023/0094090 A1
(43) Pub. Date: Mar. 30, 2023

(54) INTRODUCER SHEATH ASSEMBLY AND METHOD OF MAKING THE SAME

(71) Applicant: Medical Components, Inc., Harleysville, PA (US)

(72) Inventor: Mark S. Fisher, Sellersville, PA (US)

(21) Appl. No.: 17/953,467

(22) Filed: Sep. 27, 2022

Related U.S. Application Data
(60) Provisional application No. 63/249,095, filed on Sep. 28, 2021.

(51) Int. Cl. A61M 25/06; B29C 70/74

(52) U.S. Cl. CPC A61M 25/0668; B29C 70/74; A61M 2207/00; B29K 2101/12

(57) ABSTRACT

The present disclosure relates to a splitable introducer sheath assembly and the method of manufacturing the same. The splitable introducer sheath assembly includes a sheath tube, a hub portion, and at least two anchors. The sheath tube includes anchor tabs separately extending from the proximal sheath end. In one embodiment. The anchors secure the respective anchor tabs within the respective first and second hub halves. Alternatively, the hub halves may include a base having a first mating surface and a cap having a second mating surface; the anchor tabs extends within the corresponding one of the base with a proximal end of the anchor tab extending beyond the first mating surface; and the first mating surface of the cap is secured to the second mating surface of the base with the proximal end of the corresponding anchor tab secured therebetween.

### CROSS-REFERENCE TO RELATED APPLICATION

This application claims the benefit of U.S. provisional application 63/249,095, filed on Sep. 28, 2021, the entire contents of which is incorporated herein by reference.

### TECHNICAL FIELD

The application relates to the field of medical devices and more particularly to vascular catheter introducer sheaths for use in insertion or implantation of catheters and the like into the vasculature of patients and method of making the introducer sheath.

### BACKGROUND

Catheters are used in numerous medical procedures. In particular, catheters are used for the introduction or removal of fluids from various venous regions and vessels throughout the body, such as for hemodialysis. The procedure by which these catheters are introduced to the body is delicate and complex. One particularly intricate challenge to catheterization is enlarging a hole in the flesh and vessel to be catheterized while minimizing blood loss and trauma to the patient. Generally, to insert a catheter in a blood vessel, the vessel is identified by aspiration with a long hollow needle such as those in accordance with the Seldinger technique. When blood enters a syringe attached to the needle, indicating that the vessel has been found, a thin guide wire is then introduced, typically through the syringe needle or other introducer device, into the interior of the vessel. The introducer device is then removed, leaving the guide wire within the vessel. The guide wire projects beyond the surface of the skin.

At this point, several options are available to a physician for catheter placement. One commonly used method of inserting the catheter into the vessel is through an introducer sheath. The introducer sheath typically comprises a thin-walled tube, which serves as a temporary conduit for the catheter that is being placed. The sheath is positioned by placing a dilator, which has a hollow passageway along its longitudinal axis, inside of the sheath and passing both the dilator and the sheath together into the vessel over the guide wire. The dilator expands the opening in the blood vessel to allow for catheter insertion into the vessel. The guide wire and dilator are then removed, leaving the thin-walled sheath in place. The catheter is then inserted into the vessel through the sheath.

In a setting where a catheter with a hub or other attachment at the proximal end of the catheter has a feature which is larger than that of the inner diameter of the sheath, it is necessary to have a tear-away sheath that can be split away from the catheter as the sheath is being removed from the patient. An example of such a tear-away, or splitable or peelable, sheath, with dilator is set forth in U.S. Pat. No. 6,796,991. By splitting the sheath along its longitudinal axis as the sheath is being removed from the patient, the inserting physician will be able to pull out the sheath in such a way that the portion removed from the patient is split, thereby not interfering with any encumbrances on the catheter. Generally, splitable sheaths are manufactured in a way that aids in the tearing of the sheath at two opposing points on the circumference of the sheath, thereby splitting the sheath into two halves separated longitudinally through the center of the sheath.

A sheath is generally constructed with a hub at its proximal end. This hub can typically provide attachment point for a handle, a mating point for a dilator, and a flat surface to aid in the prevention of blood loss or contamination. When a sheath needs to be split apart in order to be successfully withdrawn from the body while leaving the catheter in place, the hub will also have to be split apart in order to clear the catheter.

The splitable sheath also presents a challenge to its manufactures. More specifically, when the sheath is peelable, the material of the sheath tube must be easily splitable but still remain some structural rigidity and integral with the hub. During the remove of the sheath after the insertion of the catheter, it may happen that the sheath tube detaches from the hub portion or its tabs so that it stays in the blood vessel. Therefore, it is desirable that the sheath is attached firmly to the hub portion while the it keeps easy to split and peel away the sheath. The U.S. Pat. No. 7,887,733 discloses a splitable introducer sheath with at least two anchor apertures in an end portion of the sheath tube. The FIG. 1 of the U.S. Pat. No. 7,887,733 is reproduced as PRIOR ART FIG. 1. As shown in FIG. 1, when molding the hub portion, a molten resin is passed into the anchor apertures thus fasten the sheath tube to the hub portion.

In the prior art as shown by FIG. 1, the sheath tube can be connected firmly to the hub portion. However, when the catheter and thus the sheath tube have a small diameter, it is difficult to make an anchor aperture in the sheath tube that is big enough to pass through the molten resin without adversely affecting the integrity of the end portion of the sheath tube.

It is desired to provide a splitable sheath, and to provide a method of making the splitable sheath, in which the sheath tube is firmly connected to the hub portion regardless of the diameter of the sheath tube.

### SUMMARY

According to one aspect of the present invention, a splitable introducer sheath assembly comprises: a sheath tube including a proximal sheath end, a distal sheath end, an elongated tubular portion extending between the proximal sheath end and the distal sheath end, and a first anchor tab and a second anchor tab separately extending from the proximal sheath end; a hub portion fixedly connected to the proximal sheath end, having a first hub half and a second hub half and defining therebetween a proximal opening; and a first anchor securing the first anchor tab within the first hub half and a second anchor securing the second anchor tab within the second hub half.

In the one aspect of the present invention, the first anchor tab and the second anchor tab may be opposed to each other in a diametrical direction of the sheath tube.

In the one aspect of the present invention, the first anchor tab and the second anchor tab may be formed by splitting a part of the tubular portion into two substantially equal halves.

In the one aspect of the present invention, each of the first and second anchor may comprise a first arm, a second arm, and a hinge that are adapted to secure a corresponding one of the first and second anchor tabs.

In the one aspect of the present invention, each of the first and second arms may comprise complimentary mating surfaces.

In the one aspect of the present invention, each of the first and second arms may comprise complimentary locking surfaces.

In the one aspect of the present invention, each of the anchor may be closed by engaging the complimentary locking surfaces of each of the first and second arms.

In the one aspect of the present invention, each of the anchor may firmly secure a corresponding one of the first and second anchor tabs.

In the one aspect of the present invention, each of the first and second arms may comprise a plurality of exterior spacers.

In the one aspect of the present invention, the first hub half may comprises a first handle extending outwardly in a diametric direction of the sheath tube, and the second hub half comprises a second handle extending outwardly in a diametric direction of the sheath tube. The first anchor may be fixed at least partially within the first hub half and the second anchor is fixed at least partially within the second hub half.

In the one aspect of the present invention, each of the first and second anchors may further comprise at least one opening.

In the one aspect of the present invention, each of the first and second anchors may further secure onto a corresponding one of the first and anchor tabs through overmolding.

In the one aspect of the present invention, each of the first and anchor tabs may extend into the corresponding first and second anchors between the complimentary mating surfaces of the first and second arms.

In the one aspect of the present invention, the tubular portion may comprise two seams that are opposed to each other along which the sheath tube can be tore apart.

According to another aspect of the present invention, a method of manufacturing a splitable introducer sheath assembly comprises: providing a splitable sheath tube having a tubular portion and a proximal end; creating a first anchor tab and a second anchor tab by splitting a part of the tubular portion at the proximal end; locking an anchor on each of the first anchor tab and the second anchor tab; and molding a hub portion around the proximal end of the sheath tube, wherein the first and second anchors are molded at least partially within the hub portion.

In another aspect of the present invention, creating the first anchor tab and the second anchor tab may comprise splitting the part of the tubular portion at the proximal end into two substantially equal halves.

In another aspect of the present invention, the anchor may comprise a first arm, a second arm, and a hinge that are adapted to secure a corresponding one of the first and second anchor tabs.

In another aspect of the present invention, each of the first and second arms may comprise complimentary mating surfaces.

In another aspect of the present invention, each of the first and second arms may comprise complimentary locking surfaces.

In another aspect of the present invention, the anchor can be closed by engaging the complimentary locking surfaces of each of the first and second arms.

In another aspect of the present invention, locking the anchor on each of the first anchor tab and the second anchor tab may comprise making the anchor frictionally secure a corresponding one of the first and second anchor tabs.

In another aspect of the present invention, each of the first and second arms may comprise a plurality of exterior spacers.

In another aspect of the present invention, molding the hub portion may comprise molding the hub portion having a first hub half and a second hub half and defining therebetween a proximal opening, wherein the first hub half comprises a first handle extending outwardly in a diametric direction of the sheath tube, and the second hub half comprises a second handle extending outwardly in a diametric direction of the sheath tube, and each anchor is fixed at least partially within the corresponding one of the first hub half and the second hub half.

In another aspect of the present invention, molding the hub portion may comprise passing a molten resin into at least one opening of the anchor.

In another aspect of the present invention, locking the anchor on each of the first anchor tab and the second anchor tab may comprise extending each of the first and anchor tabs into the corresponding anchor between the complimentary mating surfaces of the first and second arm.

According an additional aspect of the present invention, a splitable introducer sheath assembly, comprising: a sheath tube including a proximal sheath end, a distal sheath end, an elongated tubular portion extending between the proximal sheath end and the distal sheath end, and a first anchor tab and a second anchor tab separately extending from the proximal sheath end; a hub portion fixedly connected to the proximal sheath end, having a first hub half and a second hub half and defining therebetween a proximal opening; wherein each of the first hub half and the second hub half comprises a base having a first mating surface and a cap having a second mating surface; each of the first and second anchor tabs extends within the corresponding one of the base with a proximal end of the anchor tab extending beyond the first mating surface; and the first mating surface of the cap is secured to the second mating surface of the base with the proximal end of the corresponding anchor tab secured therebetween.

In the additional aspect of the present invention, the first and second mating surfaces may be serpentine and complimentary to each other.

In the additional aspect of the present invention, the cap may be secured to the base through at least one of adhesive, welding, or molding.

In the additional aspect of the present invention, the first anchor tab and the second anchor tab may be opposed to each other in a diametrical direction of the sheath tube.

In the additional aspect of the present invention, the first anchor tab and the second anchor tab may be formed by splitting a part of the tubular portion into two substantially equal halves.

In the additional aspect of the present invention, the first hub half may comprise a first handle extending outwardly in a diametric direction of the sheath tube, and the second hub half may comprise a second handle extending outwardly in a diametric direction of the sheath tube; and the first anchor is fixed at least partially within the first hub half and the second anchor is fixed at least partially within the second hub half.

In the additional aspect of the present invention, the tubular portion may comprise two seams that are opposed to each other along which the sheath tube can be tore apart.

According to a further aspect of the present invention, a method of manufacturing a splitable introducer sheath assembly, comprising: providing a splitable sheath tube having a tubular portion and a proximal end; creating a first anchor tab and a second anchor tab by splitting a part of the tubular portion at the proximal end; molding around each of the first anchor tab and the second anchor tab to form a base of a hub half, wherein the base comprises a first mating surface, a proximal end of the corresponding one of the first and second anchor tab extends beyond the first mating surface; securing a cap with a second mating surface to the base by securing the second mating surface to the first mating surface with the proximal end of the corresponding anchor tab secured between the first and second mating surfaces.

In the further aspect of the present invention, the first and second mating surfaces may be serpentine and complimentary to each other.

In the further aspect of the present invention, the method may further comprises: further securing the cap to the base through at least one of adhesive, welding, or molding.

In the further aspect of the present invention, creating the first anchor tab and the second anchor tab may comprise splitting the part of the tubular portion at the proximal end into two substantially equal halves.

In the further aspect of the present invention, molding the hub portion may comprise molding the hub portion having a first hub half and a second hub half and defining therebetween a proximal opening, wherein the first hub half comprises a first handle extending outwardly in a diametric direction of the sheath tube, and the second hub half comprises a second handle extending outwardly in a diametric direction of the sheath tube, and each anchor is fixed at least partially within the corresponding one of the first hub half and the second hub half.

### BRIEF DESCRIPTION OF DRAWINGS

FIG. 1 is a sectional view of an introducer sheath assembly in PRIOR ART.

FIG. 2 is a perspective view of an introducer sheath assembly according to an embodiment of the present invention;

FIG. 3 is a perspective view of the anchors locked on the anchor tabs of the sheath tube according to an embodiment of the present invention;

FIGS. 4A and 4B are a perspective view and a front view of the anchor in the open configuration according to an embodiment of the present invention;

FIGS. 5A and 5B are a perspective view and a front view of the anchor in the closed configuration according to an embodiment of the present invention;

FIG. 6 is a flowchart of the method of manufacturing the splitable introducer sheath assembly according to an embodiment of the present invention;

FIG. 7 is a partially exploded sectional view of an alternative embodiment according to the present invention; and

FIG. 8 is a partially exploded isometric view of the alternative embodiment according to the present invention.

### DETAILED DESCRIPTION

In the drawings, like numerals indicates like elements throughout. Certain terminology is used herein for convenience only and is not to be taken as a limitation on the present invention. The term “distal” and “proximal” refer, respectively, to direction closer and away from the vascular insertion site on the patient. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

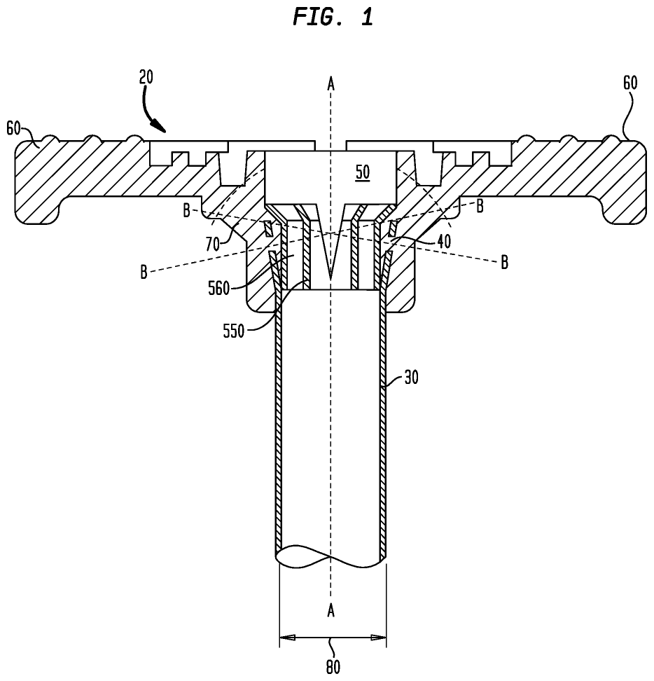

In PRIOR ART FIG. 1, the splitable sheath assembly includes a tube 30 that has a diameter 80 and a hub portion 20 coupled with the sheath tube 30. The sheath tube 30 and the hub portion 20 define a continuous lumen access. The hub portion 20 is connected to the proximal sheath end and has two handles 60 attached to the hub portion. The sheath tube 30 includes two anchor apertures 40 disposed at the proximal end of the sheath tube 30. By molding the proximal end of the sheath tube 30 together with the hub portion 20 and passing molten resin into the anchor apertures 40, a continuous mechanical connection of the sheath tube 30 and the hub portion 20 is formed.

In the prior art as shown by FIG. 1, the sheath tube can be connected firmly to the hub portion through the anchor apertures 40. However, when the catheter and thus the sheath have a small diameter, for example for pediatric use, it is difficult to make an anchor aperture in the sheath tube that is big enough to pass through the molten resin without adversely affecting the integrity of the end portion of the sheath tube.

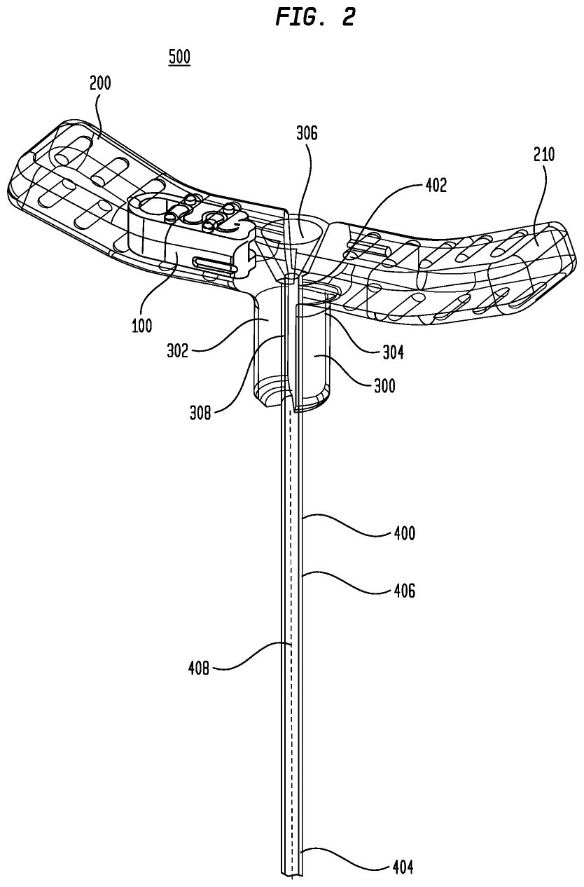

FIGS. 2-6 show embodiments of the present invention that make the sheath tube firmly connected to the hub portion regardless of the diameter of the sheath tube.

FIG. 2 is a perspective view of the splitable introducer sheath assembly 500 according to an embodiment of the invention. As shown in FIG. 2, the splitable introducer sheath assembly 500 comprises a hub portion 300 and a sheath tube 400 that is connected to the hub portion 300. The sheath tube 400 comprises a proximal sheath end 402, a distal sheath end 404, and an elongated tubular portion 406 extending between the proximal sheath end 402 and the distal sheath end 404. The hub portion 300 is fixedly attached to the sheath tube 400 at the proximal sheath end 402. More specifically, the hub portion 300 has a first hub half 302 and a second hub half 304 that are attached to or integral with each other, defining there between a proximal opening 306. The proximal opening 306 is in communication with the sheath tube 400, forming a continuous lumen in which a catheter can pass through. In this embodiment, the center part of the hub portion 300 forms a generally cylindrical part that surrounds the proximal end 402 of the sheath tube 400.

The first and second sheath halves 302 and 304 further comprise a first handle 200 and a second handles 210, respectively. The first and second handles 200 and 210 extend outwardly from the hub portion 300, and can be gripped by the practitioner when the introducer is being operated and the sheath assembly is peeled away. As shown in FIG. 2, the first and second handles 200 and 210 are opposed to each other in the lateral direction of the hub portion 300. A first anchor 100 is completely embedded within the first hub half 302 and at least partly within the first handle 200. Similarly, a second anchor (not shown) is completely embedded within the second hub half 302 and at least partly within the second handle 210. In another embodiment, the anchors may be embedded within the center cylindrical part of the hub portion. In further another embodiment, the anchors may extend partly outside of the first or second hub half and be fixed partially within the first or second hub half, as long as the respective anchors secure the respective anchor tabs of the sheath tube, which will be described in detail below, within the first and second hub halves, respectively.

As shown in FIG. 2, the hub portion 300 includes a pair of seams 308 along which the first and second hub halves 302 and 304, and are capable of separating from each other under a force exerted on the handles 200 210. The seams 308 can be formed by, such as, score lines, grooves, webbing, proliferated lines, sections that is thinner than its surrounding part, or sections that are otherwise weakened in strength. The sheath tube 400 also includes a pair of seams 408 along the tubular portion. The seams 408 can be similarly formed by score lines, grooves, proliferated lines, sections that is thinner than its surrounding part, or sections that are otherwise weakened in strength. The seams 308 align with the seams 408, and facilitate the splitting of the hub portion 300 and sheath tube 400 therealong once splitting is initiated by the practitioner after completion of the insertion of the catheter's distal portion into the vasculature of a patient (not shown).

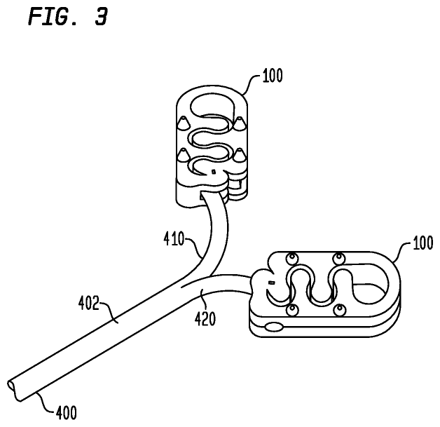

The first and second anchors 100 which fix the sheath tube 400 to the hub portion 300 are located within the first hub half 302 and the second half 304, respectively. FIG. 2 only illustrates the first anchor 100 in the first hub half 302. In the embodiment shown in FIG. 2, the first anchor 100 is embedded within the first hub half 302. Likewise, the second anchor 100 is embedded within the second hub half 302. Each of the first and second anchors 100 secures a corresponding part of the sheath tube 400 within the corresponding one of the first and second hub halves, which will be described in detail below by referring to FIG. 3. In the embodiment shown in FIG. 2, the first anchor 100 is embedded within the part of the first hub half 302 that extends to the first handle 200. Similarly, the second anchor 100 is embedded within the part of the second hub half 304 that extends to the second handle 200.

FIG. 3 is a perspective view of the sheath tube fixed to the first and second anchors 100. As shown in FIG. 3, the sheath tube 400 includes a first anchor tab 410 and a second anchor tab 420 separately extending from the proximal sheath end 402. In the embodiment shown in FIG. 3, the first and second anchor tab 410 and 420 are formed by splitting a section of the elongated tubular portion 406 at the proximal end 402 along the seams 408 into two substantially equal halves. The first anchor tab 410 and the second anchor tab 420 are opposed to each other in the diametrical direction of the sheath tube 400. In another embodiment, the anchor tabs may be formed as tabs extending from the proximal sheath end 402 by other means. For example, tabs can be molded on to the proximal sheath end 402. Tabs can also be formed by cutting and removing a portion of the material of the sheath tube 400 at the proximal sheath end 402. In addition, in another embodiment, the tabs 410 and 420 are not necessarily opposed to each other in the diametrical direction of the sheath tube 400, or substantially equal. The first anchor tab 410 is fixedly attached to the first anchor 100 and the second anchor tab 420 is fixedly attached to the second anchor 100. Therefore, when the first and second anchors 100 are attached within the hub portion 300, that is, the first and second hub halves 302 and 304, respectively, the sheath tube 400 is fixedly connected to the hub portion 300. In this embodiment, the first and second anchors 100 are identical or symmetrical to each other. However, they can be different in structure, shape, or size in another embodiment.

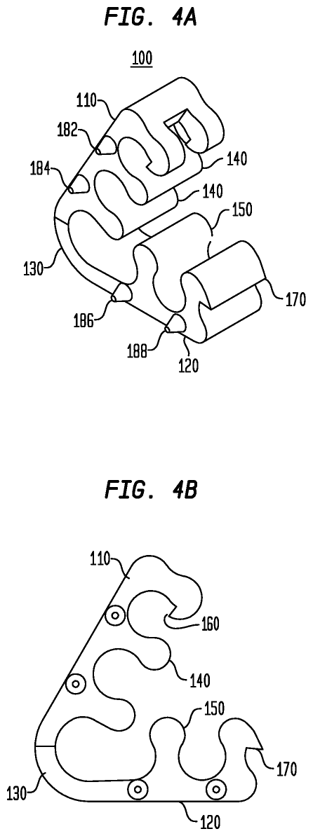

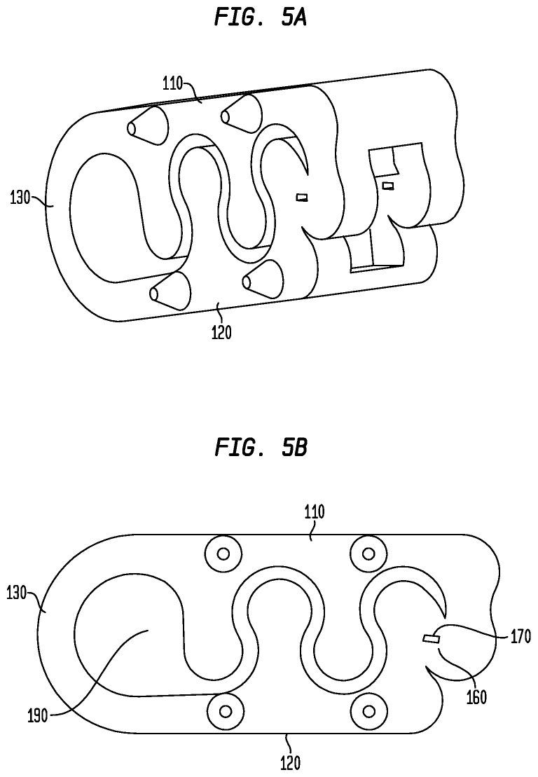

In the embodiment shown, each of the first and second anchors is formed as a clamp structure that secures the corresponding anchor tab by clamping the anchor tab. FIGS. 4A-5B depict an anchor according to an embodiment of the present invention. FIG. 4A is a perspective view of an anchor 100 in the open configuration, and FIG. 4B is a front view of an anchor 100 in the open configuration. FIG. 5A is a perspective view of an anchor 100 in the closed configuration, and FIG. 5B is a front view of an anchor 100 in the closed configuration.

As shown in FIGS. 4A-5B, an embodiment of the anchor 100 includes a first arm 110, a second arm 120, and a hinge 130. The hinge 130 connects the first arm 110 and the second arm 120 and allows the first arm 110 and the second arm 120 to pivot between the open configuration and the closed configuration. In the open configuration as shown in FIGS. 4A and 4B, the first arm 110 and the second arm 120 pivot away from each other and keep a space therebetween. In the closed configuration as shown in FIGS. 5A and 5B, the first arm 110 and the second 120 pivot towards and contact with each other. In the embodiment shown in FIGS. 4A-5B, the anchor 100 is integrally formed, and the hinge 130 is a curved section that connects the first arm 110 to the second arm 120.

The first arm 110 includes the first mating surface 140 that faces the second arm 120 while the second arm 120 includes the second mating surface 150 that faces the first arm 110. The first mating surface 140 and the second mating surface 150 are complimentary to each other so as to engage with each other in the closed configuration. Specifically, the first mating surface 140 and the second mating surface 150 are corrugated or serpentine. The first and second mating surfaces 140 and 150 may increase the contacting area between the first and second arm 110 and 120 in the closed configuration and between the arms 110 and 120 and the anchor tab 410 or 420 when the anchor tab is clamped between the first and second arms 110 and 120. Therefore, the anchor tab 410 or 420 may be firmly secured by the anchor 100.

The first arm 110 further comprises a first locking surface 160 at the end of the first arm 110, while the second arm 120 further comprises a second locking surface 170 at the end of the second arm 120. The anchor 100 can be closed by engaging the first and second locking surfaces 160 and 170. More specifically, the first locking surface 160 and the second locking surface 170 are complimentary to each other and engage with each other to keep the anchor in the closed configuration, thus stop the first arm 110 and the second arm 120 from separating from each other, against the biasing force from the hinge 120. As more clearly shown in FIG. 3, the anchor tab 410 or 420 extends into the first or second anchor 100 between the complimentary mating surfaces 140 and 150 of the first and second arms 110 and 120. Therefore, the anchor 100 may frictionally secure the anchor tab 410 or 420 by clamping the anchor tab 410 or 420 between the first and second arm 110 and 120 by a frictional force. In other embodiments, the anchor tabs may be secured to the anchors by other method such as using adhesives, by welding, or by engagement of a pin and a hole respectively provided on the anchor tab and anchor. After the anchor tabs are firmly locked by the anchors 100, the hub portion is out molded around the sheath tube 300, so that each of the first and second anchors 100 may be further secured within the hub portion 300.

When the first and second anchors 100 with the attached anchor tabs and sheath tube, are then fixedly attached to the first hub half 302 and the second hub half 304, respectively. The first anchor 100 secures the first anchor tab 410 with the first hub half 302 and the second anchor 100 secures the second anchor tab 420 within the second hub half 304. Therefore, the sheath tube 400 can be fixedly connected to the hub portion 300 at the proximal sheath end 402. Specifically, the hub portion, that is, the first and second hub halves 302 and 304 may be molded around the sheath tube at the proximal end 402 and the anchors 100. As shown in FIG. 5B, in the closed configuration, at least an opening 190 is formed in the anchor 100 so that the molten resin may pass through during the molding. Therefore, the resin is able to encapsulate the gaps and openings in the anchor 100. In some embodiments, the resin may further encapsulate the anchor 100 up to the sheath tube 400 to form the hub halves and handles as shown in FIG. 2.

The first arm 110 is further provided with a plurality of exterior spacers 182 and 184 protruding outwardly from the outer surface of the first arm 110. Similarly, the second arm 120 is further provided with a plurality of exterior spacers 186 and 188 protruding outwardly from the outer surface of the second arm 120. The spacers facilitate to secure the anchors 100 to the hub portion 300, and place the anchors 100 within the interior of the hub portion 300.

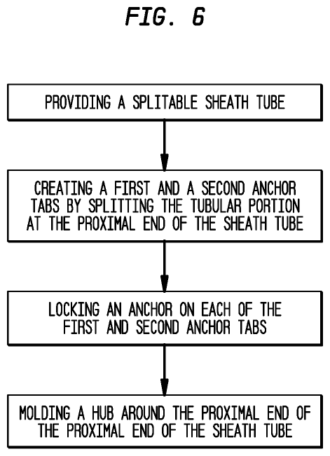

Below, a method of manufacturing a splitable introducer sheath assembly according to an embodiment of the present invention will be described by referring to FIG. 6. The method includes providing a splitable sheath tube 400 having a tubular portion 406 and a proximal end 402; creating a first anchor tab 410 and a second anchor tab 420 at the proximal end 402 of the tubular portion 406; locking an anchor 100 on each of the first anchor tab 410 and the second anchor tab 420; and molding a hub portion around the proximal end of the sheath tube 300, wherein the first and second anchors 100 are molded at least partially within the hub portion.

The step of creating the first and second anchor tabs may include splitting a part of the tubular portion 406 at the proximal end 402 into two substantially equal halves.

The step of molding the hub portion preferably includes the step of molding the hub portion 300 having a first hub half 302 and a second hub half 304 and defining therebetween a proximal opening 306, wherein the first hub half 302 comprises a first handle 200 extending outwardly in a diametric direction of the sheath tube 400, and the second hub half 304 comprises a second handle 400 extending outwardly in a diametric direction of the sheath tube 400, and each anchor 100 is fixed at least partially within the corresponding one of the first hub half and the second hub half.

Molding the hub portion 300 around the distal end of the sheath tube 400 forms a contiguous and reinforced mechanical connection among the anchors 100, the hub portion 300, and the sheath tube 400. Since the anchor 100 holds the anchor tab 410 or 420 with the mating surfaces 140 and 150, the contact area between each anchor and each anchor tab is greatly increased. Therefore, the anchor tabs and thus the sheath tub is reliably and firmly secured with the hub portion 300. In the embodiment shown by FIG. 2, the anchors are completely embedded within the hub portion 300 and extend at least part into the handles 200 after the overmolding. However, depending on the size, shape, and position of the components, the anchors may be molded within the center cylindrical part of the hub portion in another embodiment. In further another embodiment, the anchors may be out molded to be partially within the hub portion with the remaining part extending out of the hub portion.

Each of the anchor 100 may further comprise an opening 190 which may pass through the molten resin during the out molding. Compared with the opening on the anchor tab in the prior art, the opening 190 on the anchor 100 can be greater in size and thus can be more reliably attached to the molded hub portion 300.

The hub portion may be formed from an injectable thermoplastic resin.

Such a resin should be composed of a biocompatible material suitable for use in medical procedures. The resin chosen also should facilitate manufacture using, e.g., extrusion or injection molding techniques. However, it will be appreciated that the materials and manufacturing methods may vary depending on the application. The anchors and the hub may be made with the same or different material. When the anchors and the hub are made with the same material, particularly with the same color, the finished hub would appear as uniform construction. The sheath tube may also be constructed of any biocompatible material suitable for use in medical procedures. The sheath tube may be made from a thermosetting or thermoplastic resin that can be the same as or different from the hub portion's material.

Exemplary thermoplastic resins include unsaturated polyester, vinyl, vinyl ester, epoxy, urethane, phenolic, polyethylene, polypropylene, polymethyl methacrylate, polyvinyl chloride, polyamide, acrylonitrile butadiene styrene (ABS), polycarbonate, and polytetrafluoroethylene.

### Alternative Embodiment

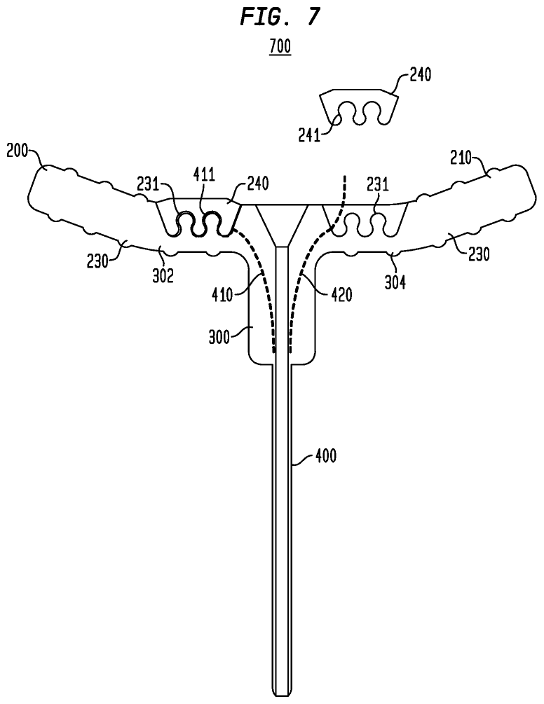

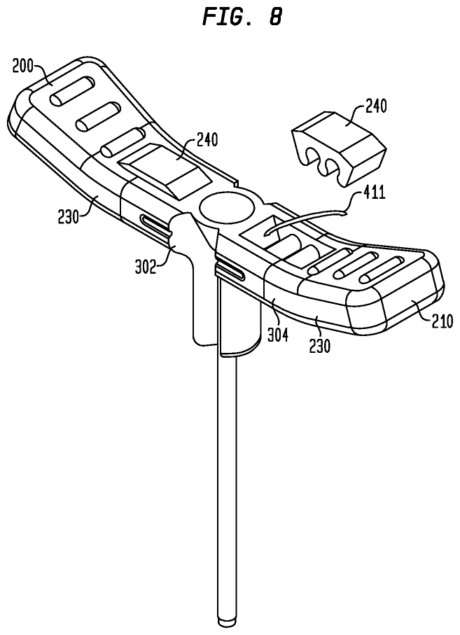

FIGS. 7-8 illustrate partially exploded sectional view and isometric view of a splitable introducer sheath assembly 700 according to an alternative embodiment of the present invention, respectively. Similar to the splitable introducer sheath assembly 500 as shown in FIG. 2, the splitable introducer sheath assembly 700 comprises a hub portion 300, a sheath tube 400 that is connected to the hub portion 300. The hub portion 300 has a first hub half 302 and a second hub half 304 each of which comprises a handle 200 or 210 extending outwardly in a diametric direction.

The splitable introducer sheath assembly 700 differs from the splitable introducer sheath assembly 500 in that each of the first and second hub half further comprises a base 230 and a cap 240. The base 230 includes a first mating surface 231 while the cap 240 includes a second mating surface 241. In the embodiment shown in FIG. 7, the first mating surface 231 and the second mating surface 241 are complimentary to each other so as to be able to engage with each other. Specifically, the first mating surface 231 and the second mating surface 241 are at least partially corrugated or serpentine.

In FIGS. 7 and 8, the first cap 240 is shown to be engaged with the first base 230 of the first hub half 302 while the second cap 240 has yet to be engaged with the first base 230 of the second hub half 304. As shown in FIG. 7, in the second hub half 304, the anchor tab 410, which is shown in broken line, is integrally molded together with the base 230 while the proximal end of the anchor tab 410 extends beyond the first mating surface 231. For example, when molding the base 230, two inserts having tapers on the perimeter may be made for each base creating a space therebetween to load the anchor tab 410 or 420 with its proximal end extending outside of the base. The loading of the anchor tab is timed when molding material has not solidified. Therefore, as the mold closes, the tab are compressed onto and become integral with the base.

Since the proximal end of the anchor tab 410 extends beyond the first mating surface 231 of the base 230, the proximal end of the anchor tab 410 can be clamped between the base 230 and the cap 240, more specifically, the first mating surface 231 and the second mating surface 241, when the cap 240 is snapped onto the base 230 through the engagement between the serpentine parts of first mating surface 231 and the second mating surface 241, which is shown in the first hub half 302. As shown in the first hub half 302, the proximal end 411 of the anchor tab 410 that is the section extending beyond the first mating surface 231 is clamped between the first mating surface 231 of the base 230 and the second mating surface 241 of the cap 240. The first mating surface 231 and the second mating surface 241 are at least partially corrugated or serpentine, so that they can accommodate a relative long proximal end 411 and firmly secure the proximal end 411 therebetween. In addition to the engagement between the base and the cap, or alternatively, the base 230 and the cap 240 can be secured to each other through other means, such as adhesive, welding, molding while clamping the proximal end of the anchor tab in between. In another embodiment, the base 230 and the cap 240 can be overmolded, after attached to each other with the proximal end of the anchor tab therebetween, to be integral with each other. In other embodiments, the first and second mating surfaces can be flat or of other shapes as long as they can be engaged with or otherwise secured to each other while sandwiching the proximal end 411 therebetween. The proximal end of the second anchor tab 420 can be similarly clamped and secured between the base 230 and the cap 240.

In the embodiment shown in FIG. 7, the first mating surface 231 of the base 230 is located on the top surface of the base 230. However, in other embodiment, the first mating surface 231 can be provided on the other surfaces of the base 230, such as, any of the side surface or the bottom surface. The length of the anchor tab 410 may be adjusted so as to extend beyond the first mating surface with a length sufficient to be clamped between the first mating surface 231 of the base 230 and the second mating surface 241 of the cap 240.

In the embodiment shown in FIGS. 7 and 8, the base 230 and the cap 240 are separate component. However, alternatively, the base 230 and the cap 240 can be connected or integral with each other. For example, one end of the cap 240 is attached to the base with sufficient flexibility to allow the cap 240 hinges around the end relative to the base 230. Therefore, the cap 240 and the base 230 may be molded or manufactured together as an integral component.

Although exemplary implementations of the invention have been depicted and described in detail herein, it will be apparent to those skilled in the relevant art that various modifications, additions, substitutions, and the like can be made without departing from the spirit of the invention and these are therefore considered to be within the scope of the invention as defined in the following claims.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting, but are instead exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the terms “comprising” and “wherein.” Moreover, in the following claims, the terms “first,” “second,” and “third,” are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

What is claimed:

1. A splitable introducer sheath assembly, comprising:
a sheath tube including a proximal sheath end, a distal sheath end, an elongated tubular portion extending between the proximal sheath end and the distal sheath end, and a first anchor tab and a second anchor tab separately extending from the proximal sheath end;
a hub portion fixedly connected to the proximal sheath end, having a first hub half and a second hub half and defining therebetween a proximal opening; and
a first anchor securing the first anchor tab within the first hub half and a second anchor securing the second anchor tab within the second hub half.

2. The introducer sheath assembly of claim 1, wherein the first anchor tab and the second anchor tab are opposed to each other in a diametrical direction of the sheath tube.

3. The introducer sheath assembly of claim 1, wherein the first anchor tab and the second anchor tab are formed by splitting a part of the tubular portion into two substantially equal halves.

4. The introducer sheath assembly of claim 1, wherein each of the first and second anchor comprises a first arm, a second arm, and a hinge that are adapted to secure a corresponding one of the first and second anchor tabs.

5. The introducer sheath assembly of claim 4, wherein each of the first and second arms comprises complimentary mating surfaces.

6. The introducer sheath assembly of claim 4, wherein each of the first and second arms comprises complimentary locking surfaces.

7. The introducer sheath assembly of claim 6, wherein each of the anchors can be closed by engaging the complimentary locking surfaces of each of the first and second arms.

8. The introducer sheath assembly of claim 1, wherein each of the anchors firmly secure a corresponding one of the first and second anchor tabs.

9. The introducer sheath assembly of claim 8, wherein each of the anchors frictionally secure a corresponding one of the first and second anchor tabs.

10. The introducer sheath assembly of claim 8, wherein each of the anchors secure a corresponding one of the first and second anchor tabs by any one of adhesive, welding, and engagement of a pin on one of the anchor and the corresponding anchor tab with a hole on the other of the anchor and the corresponding anchor tab.

11. The introducer sheath assembly of claim 4, wherein each of the first and second arms comprises a plurality of exterior spacers.

12. The introducer sheath assembly of claim 1, wherein
the first hub half comprises a first handle extending outwardly in a diametric direction of the sheath tube, and the second hub half comprises a second handle extending outwardly in a diametric direction of the sheath tube; and
the first anchor is fixed at least partially within the first hub half and the second anchor is fixed at least partially within the second hub half.

13. The introducer sheath assembly of claim 1, wherein
each of the first and second anchors further comprises at least one opening.

14. The introducer sheath assembly of claim 1, wherein each of the first and second anchors further secures onto a corresponding one of the first and anchor tabs through overmolding.

15. The introducer sheath assembly of claim 4, wherein each of the first and second anchor tabs extends into the corresponding anchor between the complimentary mating surfaces of the first and second arms.

16. The introducer sheath assembly of claim 1, wherein the tubular portion comprises two seams that are opposed to each other along which the sheath tube can be tore apart.

17. A method of manufacturing a splitable introducer sheath assembly, comprising:
providing a splitable sheath tube having a tubular portion and a proximal end;
creating a first anchor tab and a second anchor tab by splitting a part of the tubular portion at the proximal end;
locking an anchor on each of the first anchor tab and the second anchor tab; and
molding a hub portion around the proximal end of the sheath tube,
wherein the first and second anchors are molded at least partially within the hub portion.

18. The method of manufacturing a splitable introducer sheath assembly of claim 17, wherein creating the first anchor tab and the second anchor tab comprises splitting the part of the tubular portion at the proximal end into two substantially equal halves.

19. The method of manufacturing a splitable introducer sheath assembly of claim 17, wherein the anchor comprises a first arm, a second arm, and a hinge that are adapted to secure a corresponding one of the first and second anchor tabs.

20. The method of manufacturing a splitable introducer sheath assembly of claim 19, wherein each of the first and second arms comprises complimentary mating surfaces.

21. The method of manufacturing a splitable introducer sheath assembly of claim 19, wherein each of the first and second arms comprises complimentary locking surfaces.

22. The method of manufacturing a splitable introducer sheath assembly of claim 21, wherein the anchor can be closed by engaging the complimentary locking surfaces of each of the first and second arms.

23. The method of manufacturing a splitable introducer sheath assembly of claim 17, wherein locking the anchor on each of the first anchor tab and the second anchor tab comprises making the anchor frictionally secure a corresponding one of the first and second anchor tabs.

24. The method of manufacturing a splitable introducer sheath assembly of claim 17, wherein locking the anchor on each of the first anchor tab and the second anchor tab comprises locking an anchor on each of the first anchor tab and the second anchor tab by any one of adhesive, welding, and engagement of a pin on one of the anchor and the corresponding anchor tab with a hole on the other of the anchor and the corresponding anchor tab.

25. The method of manufacturing a splitable introducer sheath assembly of claim 19,
wherein each of the first and second arms comprises a plurality of exterior spacers.

26. The method of manufacturing a splitable introducer sheath assembly of claim 17,
wherein
molding the hub portion comprises molding the hub portion having a first hub half and a second hub half and defining therebetween a proximal opening, wherein the first hub half comprises a first handle extending outwardly in a diametric direction of the sheath tube, and the second hub half comprises a second handle extending outwardly in a diametric direction of the sheath tube, and each anchor is fixed at least partially within the corresponding one of the first hub half and the second hub half.

27. The method of manufacturing a splitable introducer sheath assembly of claim 17,
wherein
molding the hub portion comprises passing a molten resin into at least one opening of the anchor.

28. The method of manufacturing a splitable introducer sheath assembly of claim 20,
wherein locking the anchor on each of the first anchor tab and the second anchor tab comprises extending each of the first and anchor tabs into the corresponding anchor between the complimentary mating surfaces of the first and second arm.

29. A splitable introducer sheath assembly, comprising:
a sheath tube including a proximal sheath end, a distal sheath end, an elongated tubular portion extending between the proximal sheath end and the distal sheath end, and a first anchor tab and a second anchor tab separately extending from the proximal sheath end;
a hub portion fixedly connected to the proximal sheath end, having a first hub half and a second hub half and defining therebetween a proximal opening;
wherein each of the first hub half and the second hub half comprises a base having a first mating surface and a cap having a second mating surface;
each of the first and second anchor tabs extends within the corresponding one of the base with a proximal end of the anchor tab extending beyond the first mating surface; and
the first mating surface of the cap is secured to the second mating surface of the base with the proximal end of the corresponding anchor tab secured therebetween.

30. The introducer sheath assembly of claim 29, wherein the first and second mating surfaces are serpentine and complimentary to each other.

31. The introducer sheath assembly of claim 29, wherein the cap is secured to the base through at least one of adhesive, welding, or molding.

32. The introducer sheath assembly of claim 29, wherein the first anchor tab and the second anchor tab are opposed to each other in a diametrical direction of the sheath tube.

33. The introducer sheath assembly of claim 32, wherein the first anchor tab and the second anchor tab are formed by splitting a part of the tubular portion into two substantially equal halves.

34. The introducer sheath assembly of claim 29, wherein
the first hub half comprises a first handle extending outwardly in a diametric direction of the sheath tube, and the second hub half comprises a second handle extending outwardly in a diametric direction of the sheath tube; and
the first anchor is fixed at least partially within the first hub half and the second anchor is fixed at least partially within the second hub half.

35. The introducer sheath assembly of claim 29, wherein the tubular portion comprises two seams that are opposed to each other along which the sheath tube can be tore apart.

36. A method of manufacturing a splitable introducer sheath assembly, comprising:
providing a splitable sheath tube having a tubular portion and a proximal end;
creating a first anchor tab and a second anchor tab by splitting a part of the tubular portion at the proximal end;
molding around each of the first anchor tab and the second anchor tab to form a base of a hub half, wherein the base comprises a first mating surface, a proximal end of the corresponding one of the first and second anchor tab extends beyond the first mating surface;
securing a cap with a second mating surface to the base by securing the second mating surface to the first mating surface with the proximal end of the corresponding anchor tab secured between the first and second mating surfaces.

37. The method of manufacturing a splitable introducer sheath assembly of claim 36,
wherein the first and second mating surfaces are serpentine and complimentary to each other.

38. The method manufacturing a splitable introducer sheath assembly of claim 36, further comprising: further securing the cap to the base through at least one of adhesive, welding, or molding.

39. The method of manufacturing a splitable introducer sheath assembly of claim 36,
wherein creating the first anchor tab and the second anchor tab comprises splitting the part of the tubular portion at the proximal end into two substantially equal halves.

40. The method of manufacturing a splitable introducer sheath assembly of claim 36,
wherein
molding the hub portion comprises molding the hub portion having a first hub half and a second hub half and defining therebetween a proximal opening, wherein the first hub half comprises a first handle extending outwardly in a diametric direction of the sheath tube, and the second hub half comprises a second handle extending outwardly in a diametric direction of the sheath tube, and each anchor is fixed at least partially within the corresponding one of the first hub half and the second hub half.
